(19) United States
(12) Patent Application Publication
(10) Pub. No.: US 2022/0351813 A1
(43) Pub. Date: Nov. 3, 2022

(54) METHOD AND APPARATUS FOR TRAINING AUTOMATIC TOOTH CHARTING SYSTEMS

(71) Applicants: TROPHY SAS, Marne La Vallee Cedex 2 (FR); CARESTREAM DENTAL LLC, Atlanta, GA (US)

(72) Inventors: Jean-Marc INGLESE, Bussy-Saint-Georges (FR); Edward SHELLARD, Atlanta, GA (US); Yannick GLINEC, Serris (FR); Xavier RIPOCHE, Marne La Vallee (FR)

(21) Appl. No.: 17/427,734

(22) PCT Filed: Feb. 7, 2020
(86) PCT No.: PCT/EP2020/053129

§ 371 Date: Aug. 2, 2021

Related U.S. Application Data
(60) Provisional application No. 62/802,679, filed on Feb. 7, 2019.

(51) Int. Cl. G16H 15/00; G16H 10/60; A61C 7/00; G16H 30/40

(52) U.S. Cl. CPC G16H 15/00; G16H 10/60; A61C 7/002; G16H 30/40

(57) ABSTRACT

At least one embodiment of a method for training automatic tooth charting systems, the method comprising: obtaining, through a communication network, a plurality of electronic dental charts generated by the plurality of tooth charting systems, the plurality of electronic dental charts being related to a plurality of patients; for each of the obtained electronic dental charts: extracting at least a portion of an image representing a tooth or a region of interest and obtaining at least a corresponding item of information characterizing the represented tooth or the represented region of interest; storing the extracted at least a portion of the image and the corresponding item of information in a training data set, training the automatic dental charting system with the training data set.

### FIELD OF THE INVENTION

The present invention relates to the technical field of tooth charting and more specifically to a method and an apparatus for training automatic tooth charting systems, for example training automatic tooth charting systems based on artificial intelligence such as neural network based tooth charting systems.

### BACKGROUND OF THE INVENTION

Dental charts aid the dental practitioner in the systematic diagnosis, tracking, and treatment of teeth and supporting structures. With a generalized use of electronic devices for image storage and display by the dental practitioners, digital dental charts that can be displayed and updated as needed are widely used.

There exist methods for automatic generation of an electronic dental chart for a patient, using information obtained from analysis of any of a number of types of digital images obtained from the patient, such as the method described in U.S. Pat. No. 8,416,984. This method makes it possible to generate a template dental chart for a patient that represents the position of each imaged tooth with a symbol characterizing a state of the tooth (e.g. restorations and treatments) according to the obtained image data.

FIG. 1a is a representation of a dental chart 100 of a patient, wherein each tooth is represented by a symbol such symbols 105. Dental chart 100 may be generated from images of the patient's teeth, using analysis of the images. It may show features that are particular to the patient, such as the number of teeth, the relative tooth sizes, and the detected tooth outlines and angles, as well as the overall tooth condition, the tooth color, and restorations and other treatments for the teeth or for specific surfaces of the teeth.

As illustrated, different types of images may be associated with the tooth representations. The images that are obtained can be of one or more image types or modalities, including visible light images (VL), ultraviolet light images (UV), infrared light images (IR), fluorescence images (F), OCT (optical coherence tomography) images, X-ray images (X), image projections used for forming a volume image in CBCT (dental cone-beam computed tomography) processing, contour images, 3D meshes images, and ultrasound images.

Tooth symbols may be appropriately highlighted or otherwise marked to indicate whether or not images have been obtained and associated with the corresponding teeth and to give specific information about the corresponding tooth such as information about previous restoration and treatments or information about the surrounding gum or surrounding bone.

Images may be added to the dental chart at any time, typically each time a patient takes an appointment with a dental practitioner. Likewise, the tooth information may be updated at any time.

As shown in FIG. 1b, the dental practitioner may use a pointing device to access images or items of information associated with a tooth, to link new images to a tooth, or to enter new items of information with regard to a tooth. For example, the dental practitioner may use pointer 110, controlled with a mouse, to open window 115 in order to edit or correct notes in relation with the tooth referenced 2 in text field 120.

While such dental charts are proven to be very efficient, there is continuous need to improve them, to improve their relevance, and to improve the way they are constructed.

### SUMMARY OF THE INVENTION

The present invention has been devised to address one or more of the foregoing concerns.

According to a first aspect of the invention, there is provided a computer method for training an automatic dental charting system connected to a plurality of dental information systems via a communication network, each of the dental information system being configured for generating electronic dental charts, each of the generated electronic dental charts comprising digital images and associated items of information characterizing at least a portion of the corresponding digital image, the method comprising:

obtaining, through the communication network, a plurality of electronic dental charts generated by the plurality of tooth charting systems, the plurality of electronic dental charts being related to a plurality of patients;

for each of the obtained electronic dental charts:

extracting at least a portion of an image representing a tooth or a region of interest and obtaining at least a corresponding item of information characterizing the represented tooth or the represented region of interest;

storing the extracted at least a portion of the image and the corresponding item of information in a training data set,

training the automatic dental charting system with the training data set.

According to the method of the invention, an automatic dental charting system may be trained to generate and/or update reliable electronic dental charts.

According to embodiments, the method further comprises filtering the image from which the at least a portion of the image is extracted.

According to embodiments, the extracted at least a portion of the image representing a tooth or a region of interest is automatically extracted based on an obtained item of information characterizing a tooth or a region of interest.

According to embodiments, the method further comprises obtaining a type of the image from which the at least a portion of the image is extracted, the obtained type of the image being stored in the training data set in relation with the corresponding at least a portion of the image.

According to embodiments, the method further comprises identifying the extracted at least a portion of an image, the at least a portion of an image being identified as a function of items of information associated with the image wherein the at least a portion of image is identified.

According to embodiments, at least one of the electronic dental charts comprises images of several types representing at least the same tooth or the same region of interest, the extracting and storing steps being repeated so that the training data set comprises at least a portion of each of the images of several types. Such types may comprise the ultraviolet image type, visible-light image type, infrared image type, OCT image type, X-ray image type, CBCT image type, ultrasound image type, fluorescence image type, and/or 3D meshes image type.

According to embodiments, the automatic dental charting system comprises an artificial intelligence engine that may comprises at least one artificial neural network.

According to embodiments, each of the electronic dental charts of the plurality of electronic dental charts are obtained from a server, through the communication network, the server being different from the dental information systems having generated the electronic dental charts of the plurality of electronic dental charts.

According to a second aspect of the invention, there is provided a computer method for automatically assigning charting data to at least a portion of an image representing a tooth or a region of interest, the method comprising:

obtaining at least a portion of an image representing a tooth or a region of interest and

assigning an item of information characterizing the represented tooth or region of interest to the at least a portion of an image using an automatic dental charting system trained according to the method described above.

At least parts of the methods according to the invention may be computer implemented. Accordingly, the present invention may take the form of an entirely hardware embodiment, an entirely software embodiment (including firmware, resident software, micro-code, etc.) or an embodiment combining software and hardware aspects that may all generally be referred to herein as a “circuit”, “module” or “system”. Furthermore, the present invention may take the form of a computer program product embodied in any tangible medium of expression having computer usable program code embodied in the medium.

Since the present invention can be implemented in software, the present invention can be embodied as computer readable code for provision to a programmable apparatus on any suitable carrier medium. A tangible carrier medium may comprise a storage medium such as a floppy disk, a CD-ROM, a hard disk drive, a magnetic tape device or a solid state memory device and the like. A transient carrier medium may include a signal such as an electrical signal, an electronic signal, an optical signal, an acoustic signal, a magnetic signal or an electromagnetic signal, e.g. a microwave or RF signal.

### BRIEF DESCRIPTION OF THE DRAWINGS

Other features and advantages of the invention will become apparent from the following description of non-limiting exemplary embodiments, with reference to the appended drawings, in which:

FIGS. 1a and 1b are representations of a dental chart of a patient;

FIG. 2 illustrates a computing device for training an automatic dental charting system, connected to a plurality of dental information systems via a communication network, wherein embodiments of the invention can be carried out;

FIG. 3 is a schematic block diagram of the computing device illustrated in FIG. 2, for implementation of one or more embodiments of the invention;

FIG. 4 is a schematic diagram illustrating a dental information system such as one of the dental information systems in FIG. 2, wherein electronic dental charts and/or portions of electronic dental charts are generated;

FIG. 5 illustrates steps of an automatic tooth charting system; and

FIG. 6a illustrates an example of steps carried out in an AI engine such as the one represented in FIG. 5 for generating electronic dental charts and FIG. 6b illustrates an example of steps for training such an AI engine.

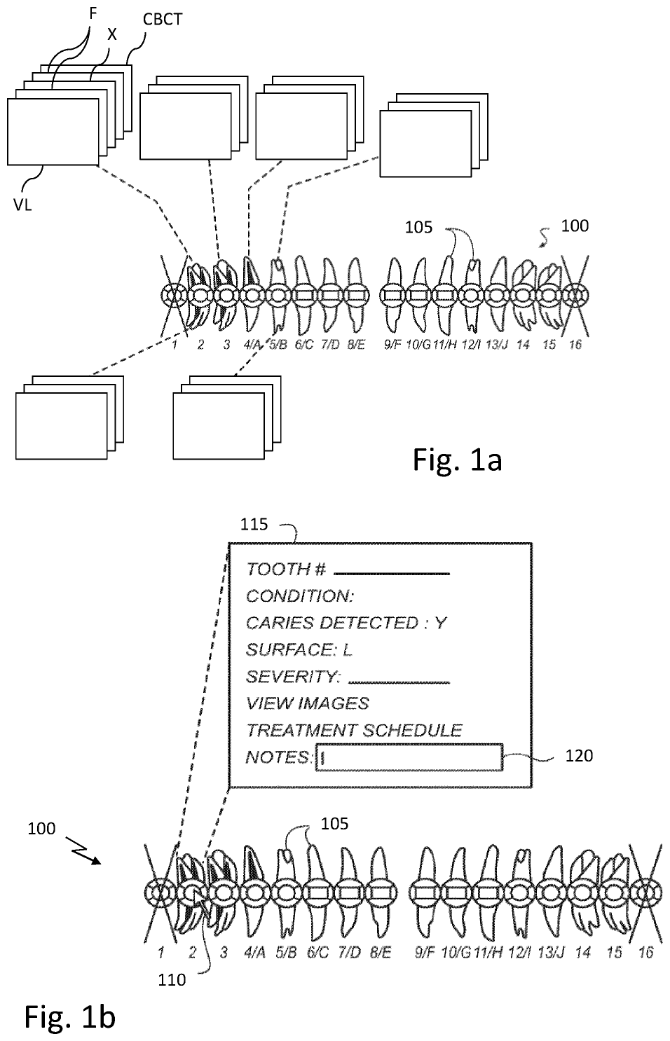

### DETAILED DESCRIPTION OF EMBODIMENTS OF THE INVENTION

The following is a detailed description of particular embodiments of the invention, reference being made to the drawings in which the same reference numerals identify the same elements of structure in each of the several figures.

In the drawings and text that follow, like components are designated with like reference numerals, and similar descriptions concerning components and arrangement or interaction of components already described are omitted. Where they are used, the terms “first”, “second”, and so on, do not necessarily denote any ordinal or priority relation, but may simply be used to more clearly distinguish one element from another.

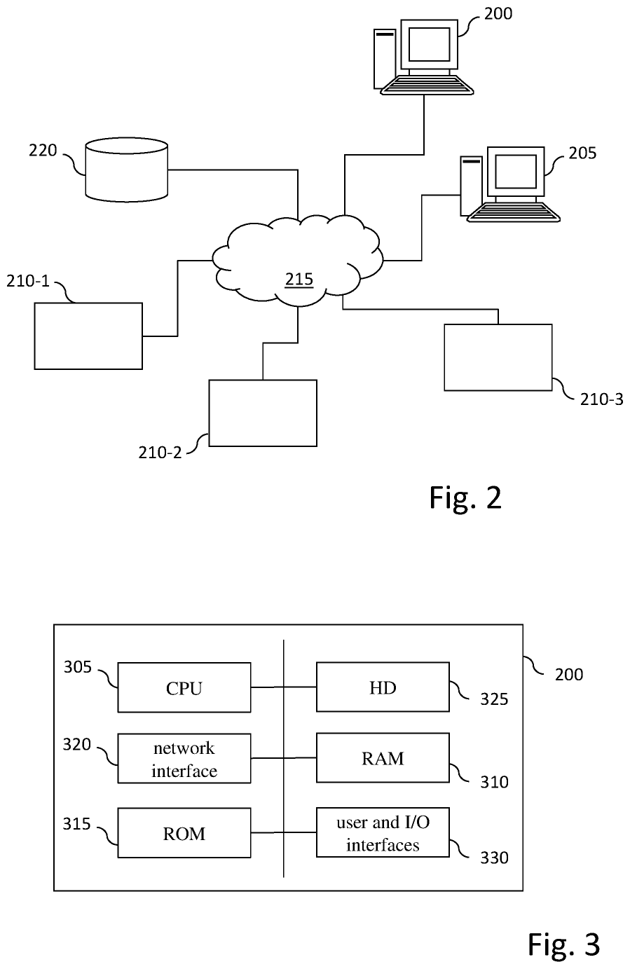

FIG. 2 illustrates a computing device 200 for training an automatic dental charting system 205, connected to a plurality of dental information systems 210-1 to 210-3 via a communication network 215, wherein embodiments of the invention can be carried out. According to the illustrated example, computing device 200 is further connected to a storage device 220.

Computing device 200 and automatic dental charting system 205 may be two different devices directly connected to each other or connected through communication network 215 or through another communication network, for example a private network. Computing device 200 and automatic dental charting system 205 may also be integrated in the same device.

It is to be noted that for the sake of illustration, only three dental information systems and one storage device are represented. However, it is to be understood that computing device 200 may be connected to several hundred or thousands of dental information systems and/or to several hundred or thousands of storage devices.

According to embodiments, each dental information system is configured for obtaining dental images of patients and for generating electronic dental charts. The obtained images may be of different types or modalities, including visible light images (VL), ultraviolet images (UV), infrared images (IR), OCT images, fluorescence images (F), X-ray images (X), image projections used for forming a volume image in CBCT (dental cone-beam computed tomography) processing, contour images, 3D meshes images, and ultrasound images. The generated electronic dental charts comprise images and associated items of information for teeth of patients, providing information about the teeth of the patients. Both images and associated items of information are contemporary.

The electronic dental charts or portions of the electronic dental charts may be obtained directly from dental information systems or from one or more storage devices, such as storage device 220, where they have been stored previously.

According to embodiments, the dental information systems generate electronic dental charts based on the same template or on compatible templates, wherein same symbols have the same meaning, so that they can be decoded automatically by a piece of software. For the sake of illustration, the electronic dental charts are generated by the same software application installed within each of the dental information systems or the same software application accessed by each of the dental information systems (for example if the software application is provided as a service).

Still according to particular embodiments, each of the dental information systems may transfer a generated electronic dental chart or a portion of a generated electronic dental chart to a remote device, for example a remote storage device or an information system, preferably after anonymizing the generated electronic dental chart (or the portion of a generated electronic dental char) so that it is not possible to identify the patient with whom the generated electronic dental chart (or the portion of a generated electronic dental char) is associated.

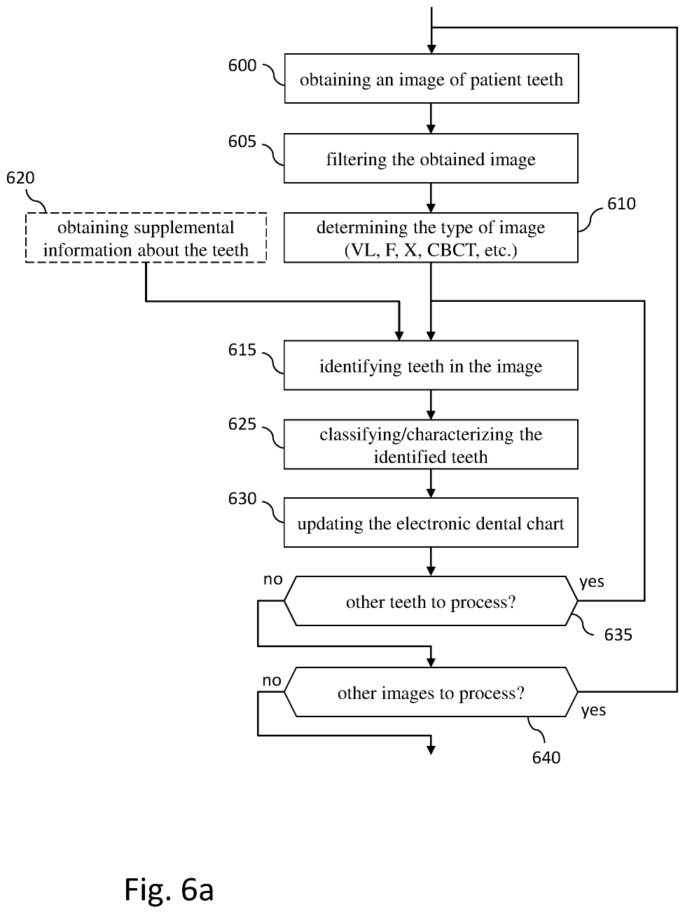

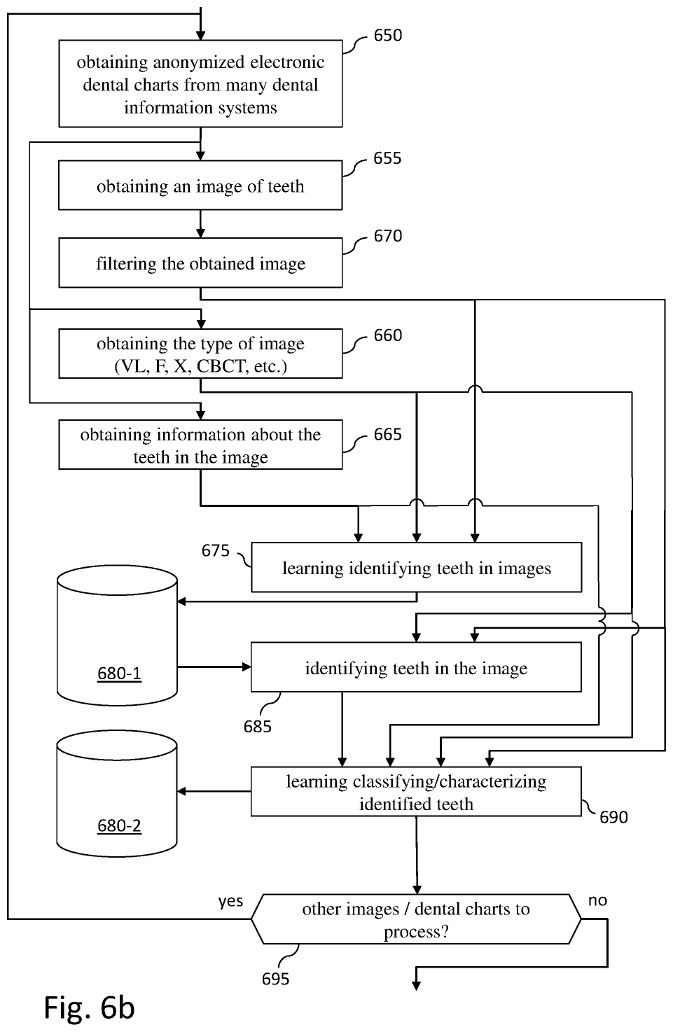

FIG. 3 is a schematic block diagram of computing device 200 illustrated in FIG. 2, for implementation of one or more embodiments of the invention, in particular for carrying out the steps or parts of the steps described by reference to FIGS. 5, 6a, and 6b.

Computing device 200 comprises a communication bus connected to:
a central processing unit 305, such as a microprocessor, denoted CPU;
a random access memory 310, denoted RAM, for storing the executable code of the method of embodiments of the invention as well as the registers adapted to record variables and parameters necessary for implementing a method for training an automatic tooth charting system according to embodiments of the invention, the memory capacity of which can be expanded by an optional RAM connected to an expansion port for example;
a read-only memory 315, denoted ROM, for storing computer programs for implementing embodiments of the invention;
a user interface and/or an input/output interface 330 which can be used for receiving inputs from a user, displaying information to a user, and/or receiving/sending data from/to external devices; and
a network interface 320 typically connected to a communication network over which digital data can be transmitted or received for receiving/sending data from/to remote devices, in particular from dental information systems 210-1 to 210-3 and/or storage device 220 and to automatic dental charting system 205. The network interface 320 can be a single network interface, or composed of a set of different network interfaces (for instance wired and wireless interfaces, or different kinds of wired or wireless interfaces). Data packets are written to the network interface for transmission or are read from the network interface for reception under the control of the software application running in the CPU 305.

Optionally, the communication bus of computing device 300 may be connected to a hard disk 325 denoted HD used as a mass storage device.

The executable code may be stored either in read-only memory 315, on hard disk 325 or on a removable digital medium such as for example a disk. According to a variant, the executable code of the programs can be received by means of a communication network, via the network interface 320, in order to be stored in one of the storage means of the computing device 200, such as hard disk 325, before being executed.

Central processing unit 305 is adapted to control and direct the execution of the instructions or portions of software code of the program or programs according to embodiments of the invention, the instructions being stored in one of the aforementioned storage means. After powering on, CPU 305 is capable of executing instructions from main RAM memory 310 relating to a software application after those instructions have been loaded from ROM 315 or from hard-disk 325 for example. Such a software application, when executed by CPU 305, causes the steps of the algorithms herein disclosed to be performed.

Any step of the algorithm herein disclosed may be implemented in software by execution of a set of instructions or program by a programmable computing machine, such as a PC (“Personal Computer”), a DSP (“Digital Signal Processor”) or a microcontroller; or else implemented in hardware by a machine or a dedicated component, such as an FPGA (“Field-Programmable Gate Array”) or an ASIC (“Application-Specific Integrated Circuit”).

According to embodiments, the schematic block diagram of automatic dental charting system 205 is similar to the schematic block diagram of computing device 200.

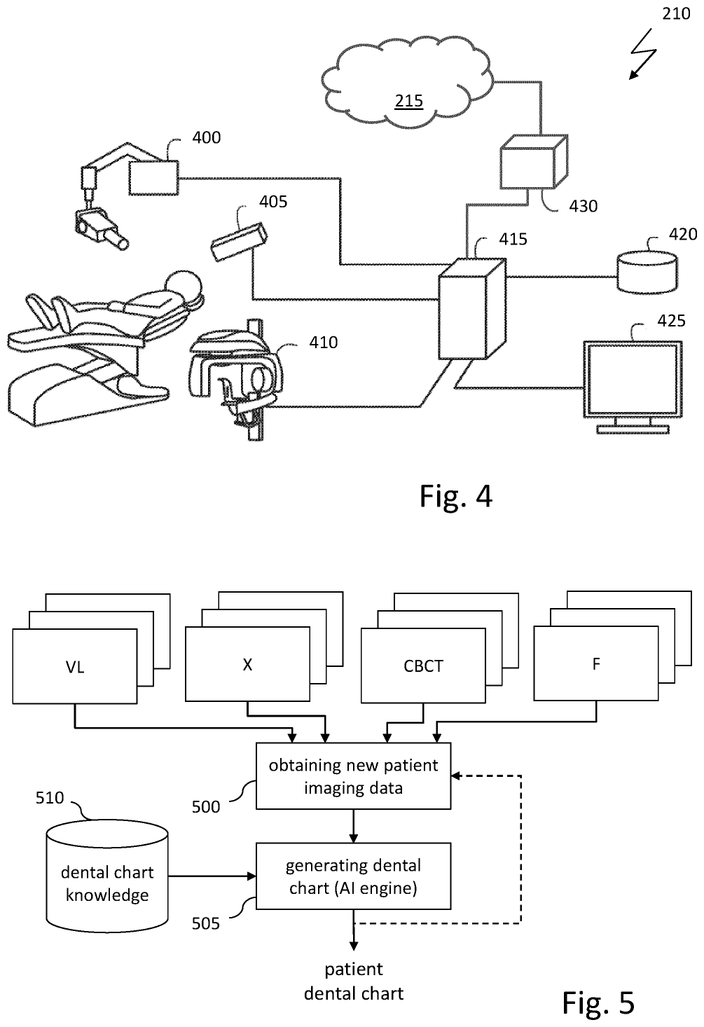

FIG. 4 is a schematic diagram illustrating a dental information system such as one of the dental information systems 210-1 to 210-3 in FIG. 2, that generates electronic dental charts and/or portions of electronic dental charts.

As illustrated, dental information system 210 includes at least one imaging apparatus, which may be an X-ray imaging apparatus 400, a digital camera 405 such as an intra-oral camera, or a dental cone-beam computed tomography (CBCT) system 410 for generating volume images of tooth structure. Other types of imaging apparatus could also be employed for obtaining images of teeth and supporting structures, gums, and related tissue, such as apparatus using ultrasound or other imaging type. In addition, various types of diagnostic measurement instrumentation may also be provided for working with dental information system 210.

Still referring to FIG. 4, a host processor 415, such as a computer or other type of dedicated logic processor for obtaining, processing, and storing image data from the imaging apparatus is also part of dental information system 210, along with one or more displays 425 for viewing image results. Host processor 415 is in data communication with one or more image capture devices and, optionally, with any number of automated measurement devices. In addition, host processor 415 can also be in data communication with a database of patient records, stored internally or on a networked host or server, for example a computer-accessible memory 420 is also provided, which may be a non-volatile memory storage device used for longer term storage, such as a device using magnetic, optical, or other data storage media.

In addition, host processor 415 comprises a network interface 430 typically connected to communication network 215 over which digital data can be transmitted or received for receiving/sending data from/to remote devices, in particular from/to computing device 200, automatic dental charting system 205, and/or storage device 220. The network interface 430 can be a single network interface, or composed of a set of different network interfaces (for instance wired and wireless interfaces, or different kinds of wired or wireless interfaces).

It is to be noted that although different dental information systems may use similar imaging apparatuses, for example similar X-ray imaging apparatuses or similar intra-oral cameras, these apparatuses may come from different manufacturers or may be set according to different settings. As a consequence, the images that are generated by these apparatuses may look different even if they represent the same object (e.g. the same tooth). Therefore, portions of electronic dental charts generated by different dental information systems, that should be similar, may actually be slightly different.

FIG. 5 illustrates steps of an automatic tooth charting system.

As illustrated, a first step (step 500) is directed to obtaining images of teeth of a patient, for example one extra-oral panoramic image and several images of one or several teeth, for example X-ray images, visible light images, ultraviolet light images, infrared light images, OCT images, fluorescence images, and CBCT images.

These images are then processed to generate an electronic dental chart (step 505) using dental chart knowledge (referenced 510). Processing of the obtained images may be based on an artificial intelligence (AI) engine comprising, for example, one or more artificial neural networks. The artificial neural network may be a supervised neural network based on supervised learning. Supervised learning requires presenting the neural network with a training set of input samples and associated labels (each label represents a target for the output). The set of corresponding labels may be determined according to prior classification performed separately from the neural network by an expert. Dental chart knowledge may be encoded in the AI engine as sets of parameter values (e.g. number of layers and nodes, weight values, etc.).

According to embodiments, an extra-oral panoramic image is used to generate a basic electronic dental chart that is supplemented by other images, of different types, and by items of information characterizing the teeth represented on the images.

As suggested with the dotted arrow, an electronic dental chart may be updated automatically at any time by processing new images and/or by processing previously processed images further to new learning step (i.e. in view of new knowledge).

FIG. 6a illustrates an example of steps carried out in an AI engine such as the one represented in FIG. 5 for generating electronic dental charts and FIG. 6b illustrates an example of steps for training such an AI engine.

The steps illustrated in FIG. 6a may be carried out in an automatic dental charting system such as automatic dental charting system 205 in FIG. 2 and the steps illustrated in FIG. 6b may be carried out in the same system or in a different system, for example in computing device 200 in FIG. 2.

The AI engine may be based on a particular AI technology, for example on fuzzy logic or on artificial neural networks, a combination of AI technologies, or a combination of AI technologies and traditional technologies, for example a combination of neural networks and predetermined rules.

After an image to be processed for generating or updating an electronic dental chart has been obtained (step 600), the image is preferably filtered (step 605). Such a filtering step may comprise, for example, a step of normalizing the obtained image so that pixel values are coded on a predetermined number of bytes (e.g. 3 bytes, 1 byte per component), according to a predetermined format (e.g. YUV), according to a predetermined resolution. In addition, the normalization step may comprise a step of adjusting parameters such as luminance and range expansion. The filtering step may also comprise an image treatment such as image enhancement and image smoothing.

Next, the type of the obtained image is determined (step 610). It is preferably determined automatically, for example by identifying its source or parameters associated with the obtained image or by analyzing the latter according to well-known methods.

According to particular embodiments, the step of filtering the obtained image is carried out after determination of its type so that the filtering is adapted to the type of the image.

Next, the teeth are identified in the obtained image (step 615) so as to determine the number of represented teeth in the image, their location in the image, their size, and their shape. Such a step can be carried out by using the AI engine, after it has been trained, with or without pre-processing steps such as a segmentation step. According to embodiments, the teeth are indexed or numbered using a global index so that a link may be established between a tooth represented in an image and the same tooth represented in another image. Such global index may be the same as the one used in the known electronic dental charts.

According to embodiment, tooth identification and numbering is carried out by using bounding boxes (e.g. rectangular boxes or boxes having more precise contours). It may comprise a rough image segmentation by identifying rectangular boxes on a panoramic image so as to identify the tooth number contained in each.

Such bounding boxes may be determined by using implicit rules of an AI engine, for example by using portions of the obtained image as input of an artificial neural network. The latter, after an appropriate training, is able to identify representation of tooth and thus, to determine bounding boxes around the identified teeth.

In addition, the segmentation may implement explicit rules such as the following to disambiguate ambiguous situations (if needed):
“rectangular boxes may have some overlap which should not exceed a predefined distance (e.g. 1 mm)”, and
“the center of rectangular boxes corresponding to one arch should describe a curve which has a small spatial variation (i.e. the teeth are mostly aligned along a line or a curve with little curvature)”.

Likewise, the tooth numbering algorithm may implement implicit or explicit rules such as the following:
“on a panoramic image in occlusion, tooth numbers follow a natural predefined sequence”,
“teeth from the lower jaw can't appear on the upper jaw”, and
“the upper jaw consists in a subset of boxes, placed above a lower jaw consisting in the complimentary group of boxes”.

It is noted that information about tooth location may be provided by other means than image analysis, in particular when the obtained image comprises a subset of the teeth of a patient from which it is difficult if not impossible to derive the location of a tooth in the patient mouth. For example, it may be obtained from a user or from instructions given to the user when taking the image (step 620).

Next, the identified teeth are classified or characterized (step 625).

Such a classification/characterization step is preferably carried out by using knowledge acquired from obtained electronic dental charts during a learning phase, for example by using an isolated tooth representation as the input of a previously trained artificial neural network, used for classifying tooth representation. The output of the artificial neural network is one or more items of information characterizing the isolated tooth representation.

Again, such a step can be supplemented by using a set of rules and threshold such as the following:
“a strong saturated signal is probably a signature of metal”,
“a feature detecting metal increases the likelihood of having an amalgam, an implant, a metallic crown, a metallic bracket, a root canal, etc.”, and
“the depth of the root in the bone determines if there is a bone loss which may lead to a tooth loss”.

An object of the classification/characterization step is to associate one or more items of information to the identified teeth. Such items of information may be, for example, the following:
regarding the root and portions that are under the crown
   tooth present or not,
   periapical lesion,
   root canal or not,
   post in root,
   bone loss,
   ankyloses,
   sinus infection, and
   sinus floor swelling,
regarding portions at the crown level
   restoration or not,
   3D medial-distal extent of restoration (difficult to determine buccal-lingual extent),
   crown and type of material (porcelain, porcelain-fused metal, all metal),
   liner under restoration (shows as different grayscale in radiographs from amalgam or composite),
   decay under restoration, and
   interproximal decay,
regarding features that cannot be detected in radiograph
   buccal-lingual extent of restoration,
   fractures,
   leakage around restoration, and
   enamel fracturing around restoration.

Naturally, other items of information may be associated with teeth and/or groups of teeth.

It is also to be noted that the classification/characterization step may include some pre-processing, for example to scale and align the signal contained in each bounding box onto a normalized average tooth representation. This may help defining placement information such as mesial/distal, occlusal/buccal relative to the average tooth representation.

Once a tooth has been classified/characterized, the corresponding electronic dental chart is generated (if the current tooth is the first one of a first image associated with a new patient) or updated (step 630) accordingly. According to embodiments, the image is stored (if not already stored) and the items of information associated with the current tooth are stored in relation with this tooth, for example using its global index.

These steps are repeated for all the teeth identified in the obtained image, for all the images to be processed (steps 635 and 640).

As described above, the used AI engine may use artificial neural networks such as convolutional neural networks and deep learning technologies for localization tasks and/or classification and characterization tasks.

The process described by reference to FIG. 6a may be carried out when creating an electronic dental chart, typically from a set of images of the teeth of a patient, and each time new images of the teeth of the patient are taken or a dental practitioner intervenes on and/or monitors the patient teeth.

According to embodiments, training an AI engine such as the one described by reference to FIGS. 5 and 6a, used for identifying and/or classifying teeth, is done by using a huge amount of electronic dental charts that comprise a low level of error (these errors being typically directed to image interpretation). These electronic dental charts are preferably anonymized before or after being transmitted for being used for training the automatic dental charting system.

For the sake of reliability, the electronic dental charts to be used for training the automatic dental charting system are preferably electronic dental charts that have been generated by practitioners, controlled by practitioners, or automatically generated by reliable systems, in different locations, using different dental information systems. They share a common format making it possible for an information system to automatically obtained images and items of information associated with teeth represented in these images.

The electronic dental charts may be obtained from dental information systems and/or from servers wherein electronic dental charts generated by dental information systems are stored.

After at least one electronic dental chart has been obtained (step 650), it is analyzed to obtain an image representing teeth of a patient (step 655), to obtain a type of the obtained image (step 660), and to obtain items of information regarding the teeth represented in the obtained image (step 665).

The steps of obtaining an image, a type of an image, and items of information regarding represented teeth are carried out according to the electronic format of the electronic dental charts, that is advantageously predetermined so that data may be gathered appropriately.

It is to be noted that several images of the same tooth or of the same region of interest may be used, provided that the corresponding item of information characterizes the tooth or region of interest represented in the considered image. In other words, the item of information and the image are contemporary (i.e. a status of a tooth or a region of interest remains unchanged between the time when an image representing the tooth or the region of interest was acquired and the time when the corresponding item of information is defined).

According to particular embodiments, the obtained image is filtered (step 670). Like the filtering step described by reference to FIG. 6a, that may be carried out when generating or updating an electronic dental chart, this filtering step may comprise, for example, a step of normalizing the obtained image so that pixel values are coded on a predetermined number of bytes (e.g. 3 bytes, 1 byte per component), according to a predetermined format (e.g. YUV), according to a predetermined resolution. In addition, the normalization step may comprise a step of adjusting parameters such as luminance and range expansion.

Again, according to particular embodiments, the step of filtering the obtained image may depend on the image type.

Next, according to the illustrated example, a first learning phase is carried out to teach the AI engine how to identify representations of teeth (step 675). To that end, the obtained items of information associated with a tooth are obtained to identify the portion of the obtained image corresponding to this tooth. Such items of information may be combined with segmentation information to identify the portion of image corresponding to this tooth.

This portion of image is added to the training data set referenced 680-1, used for training the AI engine. According to embodiments, the type of image may be also be stored in the training data set, in association with the corresponding image portion.

Next, the tooth represented in the obtained image are identified (step 685), using the AI engine (after it has been trained).

Next, a second learning phase is carried out to teach the AI engine how to classify or characterize identified teeth (step 690), that is to say to enable the AI engine to associate items of information representing tooth classes and/or characteristics with tooth representations.

To that end, the obtained items of information associated with an identified tooth are obtained. The portion of image corresponding to the identified tooth and associated items of information are added to the training data set referenced 680-2, used for training the AI engine. According to embodiments, the type of image may be also be stored in the training data set, in association with the corresponding image portion.

These steps are preferably repeated for all the teeth identified in the obtained image, for all the images of all the electronic dental charts (step 695).

According to particular embodiments, some of the electronic dental charts may be removed from the set of electronic dental charts to be used for training the AI engine so as to be used for testing the AI engine after the learning phases have been carried out. In such a case, the efficiency of the automatic charting system may be determined by comparing the output of the automatic charting system with the expected response (i.e. items of information of the electronic dental charts). Such an evaluation can also be carried out during the learning phase so that it can be stopped as soon as a level of efficiency has been reached.

It is noted that the training data set(s) may be updated on a regular basis, for example each week or each month, with new electronic dental charts or with updated electronic dental charts, obtained from dental information systems. According to embodiments, the automatic tooth charting system is trained after the training data set has been updated.

While the invention has been illustrated and described in detail in the drawings and foregoing description, such illustration and description are to be considered illustrative or exemplary and not restrictive, the invention being not restricted to the disclosed embodiment. Other variations on the disclosed embodiment can be understood and performed by those skilled in the art, in carrying out the claimed invention, from a study of the drawings, the disclosure and the appended claims.

Such variations may derive, in particular, from combining embodiments as set forth in the summary of the invention and/or in the appended claims.

In particular, it is to be noted that although image analysis is directed to identification of teeth with which items of information are associated, for the sake of illustration, image analysis may be directed to identification of any regions of interest (ROI) with which items of information are to be associated, making it possible, for example, to associate a pathology of the cyst type with a representation of a portion of the jaw. In such a case, a region of interest may be identified and/or extracted, at least partially, according to an item of information, for example according to a representation of a cyst and/or according to features characterizing a cyst.

In the claims, the word “comprising” does not exclude other elements or steps, and the indefinite article “a” or “an” does not exclude a plurality. A single processor or other unit may fulfil the functions of several items recited in the claims. The mere fact that different features are recited in mutually different dependent claims does not indicate that a combination of these features cannot be advantageously used. Any reference signs in the claims should not be construed as limiting the scope of the invention.

1. A computer method for training an automatic dental charting system connected to a plurality of dental information systems via a communication network, each of the dental information system being configured for generating electronic dental charts, each of the generated electronic dental charts comprising digital images and associated items of information characterizing at least a portion of the corresponding digital image, the method comprising:
obtaining, through the communication network, a plurality of electronic dental charts generated by the plurality of tooth charting systems, the plurality of electronic dental charts being related to a plurality of patients;
for each of the obtained electronic dental charts:
extracting at least a portion of an image representing a tooth or a region of interest and obtaining at least a corresponding item of information characterizing the represented tooth or the represented region of interest;
storing the extracted at least a portion of the image and the corresponding item of information in a training data set,
training the automatic dental charting system with the training data set.

2. The method of claim 1, further comprising filtering the image from which the at least a portion of the image is extracted.

3. The method according to claim 1 or claim 2, wherein the extracted at least a portion of the image representing a tooth or a region of interest is automatically extracted based on the corresponding item of information characterizing the represented tooth or the represented region of interest.

4. The method of any one of claims 1 to 3, further comprising obtaining a type of the image from which the at least a portion of the image is extracted, the obtained type of the image being stored in the training data set in relation with the corresponding at least a portion of the image.

5. The method of any one of claims 1 to 4, further comprising identifying the extracted at least a portion of an image, the at least a portion of an image being identified as a function of items of information associated with the image wherein the at least a portion of image is identified.

6. The method of any one of claims 1 to 5, wherein at least one of the electronic dental charts comprises images of several types representing at least the same tooth, the extracting and storing steps being repeated so that the training data set comprises at least a portion of each of the images of several types.

7. The method of claim 6, wherein the several types comprise ultraviolet image type, visible-light image type, infrared image type, OCT image type, X-ray image type, CBCT image type, ultrasound image type, fluorescence image type, and/or 3D meshes image type.

8. The method of any one of claims 1 to 7, wherein the automatic dental charting system comprises an artificial intelligence engine.

9. The method of claim 8, wherein the artificial intelligence engine comprises at least one artificial neural networks.

10. The method of any one of claims 1 to 9, wherein each of the electronic dental charts of the plurality of electronic dental charts are obtained from a server, through the communication network, the server being different from the dental information systems having generated the electronic dental charts of the plurality of electronic dental charts.

11. A method for automatically assigning charting data to at least a portion of an image representing a tooth or a region of interest, the method comprising:
obtaining at least a portion of an image representing a tooth or a region of interest and
assigning an item of information characterizing the represented tooth or region of interest to the at least a portion of an image using an automatic dental charting system trained according to the method of any of claims 1 to 9.

12. A computer program product for a programmable apparatus, the computer program product comprising instructions for carrying out each step of the method according to any one of claims 1 to 11 when the program is loaded and executed by a programmable apparatus.

13. An apparatus comprising means for computer carrying out each step of the method according to any one of claims 1 to 10.

14. An automatic dental charting system trained according to the method of any of claims 1 to 9.
